(19) United States
(12) Patent Application Publication
(10) Pub. No.: US 2024/0199505 A1
(43) Pub. Date: Jun. 20, 2024

(54) SOIL AMENDMENT FOR SOLUBILIZING SILICATE IN SOIL

(71) Applicant: NORTHERN HEMP SPECIALISTS LTD., Toronto (CA)

(72) Inventor: Gregory MARSH, Toronto (CA)

(21) Appl. No.: 18/287,083

(22) PCT Filed: Apr. 13, 2022
(86) PCT No.: PCT/IB2022/053488

§ 371 Date: Oct. 16, 2023

Related U.S. Application Data
(60) Provisional application No. 63/259,895, filed on Apr. 16, 2021.

(51) Int. Cl. C05F 11/08; A01N 25/26; A01N 63/22; A01P 21/00; C05G 5/12; C05G 5/35

(52) U.S. Cl. CPC C05F 11/08; A01N 25/26; A01N 63/22; A01P 21/00; C05G 5/12; C05G 5/35

(57) ABSTRACT

The present disclosure provides a soil amendment composition for solubilizing silicate in soil. The soil amendment includes at least one a microorganism capable of solubilizing the silicate, and may further comprise at least one nutrient, binding agent or silica. This combination facilitates a slow release of silicate into the soil. Addition of the soil amendment composition encourages growth of plants, such as Cannabis plants; and may enable plants to better withstand drought, frost winds and other environmental conditions. The present disclosure provides a soil amendment for providing bioavailable silicon that may be applied infrequently.

### FIELD

The present specification is directed soil amendments for agricultural and horticultural uses and more particularly soil amendments for solubilizing silicate.

### BACKGROUND

Fertilizers are widely used to increase crop production. Organic fertilizers such as manure, compost, bone meal, or guano are derived directly from plant or animal sources. Inorganic fertilizers such as ammonium sulfate or ammonium phosphate may be manufactured, although many are derived from naturally occurring mineral deposits. Fertilizers improve crop growth by introducing or replenishing nutrients in the soil. Nutrients from the fertilizer are dissolved by water in the soil and absorbed by plants. 6 macronutrients are considered to be essential for sustainable crop yields—nitrogen, phosphorus, potassium, sulfur, calcium, and magnesium—however, silicon is known to enhance crop yield under certain growing conditions.

Silicon is continuously leached out of the soil in a process known as desilication. Furthermore, intensive cultivation practices and continuous monocultures can deplete the silicon content of soil. Silicon supplements for crops are commercially available in concentrated solutions. Typically, the silicon is provided as potassium silicate or silicon dioxide that is suspended or solubilized in water, but some silicon supplements are available as a soluble powder. A major drawback of using soluble forms of silicon is they contain relatively low concentrations of silica and easily leach out of the soil. Therefore, these forms of silicon must be continually reapplied to the soil. However, insoluble forms of silicon such as silicate minerals are not bioavailable and therefore do not provide benefits to the plants. The present disclosure addresses this gap by providing a soil amendment that includes silicate-solubilizing microorganisms. The microorganisms break down the silicate into soluble forms of silicon that can be absorbed by plants.

### SUMMARY

It is an aspect of the present disclosure to provide a soil amendment for solubilizing silicate in soil.

The above aspects can be attained by a soil amendment comprising at least one microorganism that is capable of solubilizing silicate.

The at least one microorganism may include one or more species of the genus Pseudomonas, such as Pseudomonas putida and Pseudomonas montelli. The microorganism may further include one or more species of the genus Bacillus, such as Bacillus subtilis and Bacillus amyloliquefaciens. The microorganism may further include one or more species of the genus Glomus, such as Glomus intraradices, Glomus mosseae, Glomus aggregatum, Glomus etunicatum, Glomus clarum, Glomus monosporum, and Glomus deserticola. The microorganism may further include one or more species of the genus Gigaspora, such as Gigaspora margarita. The microorganism may further include one or more species of the genus Paraglomus, such as Paraglomus brasilianum. The microorganism may further include one or more species of the genus Rhizopogon, such as Rhizopogon villosullus, Rhizopogon luteolus, Rhizopogon amylopogon, and Rhizopogon fulvigleba. The microorganism may further include one or more species of the genus Pisolithus, such as Pisolithus tinctorius. The microorganism may further include one or more species of the genus Laccaria, such as Laccaria bicolor and Laccaria laccata. The microorganism may further include one or more species of the genus Suillus, such as Suillus granulates and Suillus punctatpies. The microorganism may further include one or more species of the genus Bacillus, such as Bacillus pumilus and Bacillus firmus. The microorganism may further include one or more species of the genus Paenibacillus, such as Paenibacillus macerans and Paenibacillus macqariensis.

The soil amendment may be an aqueous solution with the microorganism(s) suspended in the aqueous solution.

The soil amendment may include at least one nutrient.

The soil amendment may include a binding agent.

The aqueous solution containing a suspension of the microorganism may be lyophilized to obtain a powder. The powder may be compounded to obtain a pellet.

The soil amendment may further include silica, particularly insoluble silica.

The microorganism and the silica may increase the potency of any nutrients added to the soil.

It is a further aspect of the present disclosure to provide a use for the above-described soil amendment to improve growth of plants.

It is a further aspect of the present disclosure to provide a use for the above-described soil amendment to improve growth of tomato plants.

It is a further aspect of the present disclosure to provide a use for the above-described soil amendment to improve vascular development of plants.

It is a further aspect of the present disclosure to provide a use for the above-described soil amendment to increase nutrient absorption by plants.

It is a further aspect of the present disclosure to provide a use for the above-described soil amendment to improve frost resistance of plants.

It is a further aspect of the present disclosure to provide a use for the above-described soil amendment to improve drought resistance of plants.

It is a further aspect of the present disclosure to provide a use for the above-described soil amendment to improve pest resistance of plants.

It is a further aspect of the present disclosure to provide a use for the above-described soil amendment to reduce transplant shock of plants.

It is a further aspect of the present disclosure to provide a use for the above-described soil amendment to improve growth of Cannabis plants.

It is a further aspect of the present disclosure to provide a use for the above-described soil amendment to improve vascular development of Cannabis plants.

It is a further aspect of the present disclosure to provide a use for the above-described soil amendment to increase nutrient absorption by Cannabis plants.

It is a further aspect of the present disclosure to provide a use for the above-described soil amendment to improve frost resistance of Cannabis plants.

It is a further aspect of the present disclosure to provide a use for the above-described soil amendment to improve drought resistance of Cannabis plants.

It is a further aspect of the present disclosure to provide a use for the above-described soil amendment to improve pest resistance of Cannabis plants.

It is a further aspect of the present disclosure to provide a use for the above-described soil amendment to increase the number of bud sites of Cannabis plants.

It is a further aspect of the present disclosure to provide a use for the above-described soil amendment to reduce the number of larf buds of Cannabis plants.

It is a further aspect of the present disclosure to provide a use for the above-described soil amendment to increase crop yield in Cannabis plants.

It is a further aspect of the present disclosure to provide a use for the above-described soil amendment to reduce transplant shock of Cannabis plants.

It is a further aspect of the present disclosure to provide a use for the above-described soil amendment to improve growth of hemp plants.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

The renderings and images are included for illustrative and interpretive purposes relative to specific embodiments and applications and should not be construed as the sole positioning, configurations, or singular use of the present invention. These embodiments are described in sufficient detail to enable those skilled in the art to practice the invention. Other embodiments may be utilized, and structural and logical changes may be made, without departing from the scope of the present invention.

In addition, the materials, methods, and examples are illustrative only, and are not intended to be limiting. In the following detailed description, numerous specific details are provided, such as the identification of various system components, to provide an understanding of embodiments of the invention. One skilled in the art will recognize, however, that embodiments of the invention can be practiced without one or more of the specific details, or with other methods, components, materials, etc. In still other instances, well-known structures, materials, or operations are not shown or described in detail to avoid obscuring aspects of various embodiments of the invention.

FIG. 1 shows a cross-section of a test group stem and a control group stem.

FIG. 2 shows a cross-section of another test group stem and another control group stem.

### DETAILED DESCRIPTION

The present disclosure is described with respect to a soil amendment for solubilizing silica.

### Definitions

“Larf” herein refers to an immature bud of a Cannabis plant, particularly a bud that is not considered commercially viable.

“Cystolith” herein refers to an outgrowth of the epidermal cell wall, usually comprising calcium carbonate.

“Epidermal fissure” herein refers to a crack in plant tissue, especially a crack caused by drought.

“Silica” herein refers to silicon dioxide (SiO₂).

“Microorganism” herein refers to any microscopic organism including bacteria, archaebacteria, fungi, protists, or viruses.

“Soluble” herein refers the state of being able to dissolve in a solvent, especially water. A substance that is soluble will separate into molecules or ions when in contact with the solvent.

“Insoluble” herein refers the state of being unable to dissolve in a solvent, especially water. A substance that that is insoluble will not separate into molecules or ions when in contact with the solvent.

“Solubilize” herein refers the process of making a substance soluble or more soluble in a solvent, especially water.

“Binding agent” herein refers to a substance that holds other materials together chemically or as an adhesive.

“Dextrose” herein refers to the sugar having the simple formula C₆H₁₂O₆, also known to as D-glucose or dextrose monohydrate.

“Tetrahydrocannabinol” and “THC” are used interchangeably herein to refer to a psychoactive cannabinoid having the simple formula C₂₁H₃₀O₂, also known as Delta-9-Tetrahydrocannabinol.

“Cannabidiol” and “CBD” are used interchangeably herein to refer to a phytocannabinoid having the simple formula C₂₁H₃₀O₂, also known as cannabidiolum.

“Cannabigerol” and “CBG” are used interchangeably herein to refer to a cannabinoid having the simple formula C₂₁H₃₂O₂.

“Cannabidivarin” and “CBDV” are used interchangeably herein to refer to a non-psychoactive cannabinoid having the simple formula C₁₉H₂₆O₂.

“Tetrahydrocannabivarin” and “THCV” are used interchangeably herein to refer to a propyl cannabinoid having the simple formula Cd₁₉H₂₆O₂.

“Cannabichromevarin” and “CBCV” are used interchangeably herein to refer to a cannabinoid having the simple formula C₁₉H₂₆O₂, also known as 2-methyl-2-(4-methylpent-3-enyl)-7-propylchromen-5-ol.

“Cannabinol” and “CBN” are used interchangeably herein to refer to a psychoactive cannabinoid having the simple formula C₂₁H₂₆O₂.

“Cannabichromene” and “CBC” are used interchangeably herein to refer to a non-psychoactive cannabinoid having the simple formula C₂₁H₃₀O₂, also known as cannabichrome, cannabichromene, pentylcannabichromene, and cannabinochromene.

“Cannabicyclol” and “CBL” are used interchangeably herein to refer to a non-psychoactive cannabinoid having the simple formula C₂₁H₂₆O₂.

“Cannabitriol” and “CBT” are used interchangeably herein to refer to a cannabinoid having the simple formula C₂₁H₃₀O₄, also known as 6,6,9-trimethyl-3-pentyl-8,10-dihydro-7H-benzo[c]chromene-1,9,10-triol.

“Soil amendment” is used herein to refer to any substance or composition that is added to soil in order to modify soil conditions.

The present disclosure provides a soil amendment for solubilizing silicate in soil which may control the release of silica into the soil. Consequently, the soil amendment may be applied infrequently.

The soil amendment comprises at least one living microorganism. The at least one microorganism may be selected for its ability to solubilize silica, however not all microorganisms capable of solubilizing silica may be suitable to apply to plants. Bacterial species of the genus Lactobacillus are capable of producing lactic acid which dissolves silica, so it was thought that adding Lactobacillus to the soil amendment would increase the rate at which silica in the soil breaks down. In fact, testing demonstrated that Lactobacillus damages the roots of at least some plants, including Cannabis. Therefore, the at least one living microorganism should be selected for its compatibility with a specific plant. In some examples, the at least one living microorganism may be selected from a group consisting of Pseudomonas putida, Pseudomonas montelli, Bacillus subtilis, Bacillus amyloliquefaciens, Glomus intraradices, Glomus mosseae, Glomus aggregatum, Glomus etunicatum, Glomus clarum, Glomus deserticola, Gigaspora margarita, Paraglomus brasilianum, Glomus monosporum, Rhizopogon villosullus, Rhizopogon luteolus, Rhizopogon amylopogon, Rhizopogon fulvigleba, Pisolithus tinctorius, Laccaria bicolor, Laccaria laccata, Suillus granulates, Suillus punctatpies, Bacillus pumilus, Paenibacillus macerans, Bacillus firmus, and Paenibacillus macqariensis. In some examples, the at least one living microorganism comprises all of the microorganisms listed above.

In some examples, each of the at least one living microorganism may comprise approximately 0.5 to 5 percent of the soil amendment by weight, although the composition of the at least one living microorganism is not particularly limited. In some examples, each of the at least one living microorganism may comprise 1.5 percent of the soil amendment by weight.

In some examples, the soil amendment may comprise a liquid suspension of the at least one living microorganism. The liquid suspension may comprise water. In examples where the soil amendment is a liquid suspension, the soil suspension may further comprise nutrients for sustaining the bacteria. The nutrients may be selected according to the nutritional needs to the at least one living microorganism. For example, the nutrients may include one or more of the following: peptides, proteins, carbohydrates, vitamins, trace elements, and minerals. In one example, the nutrients comprise 20 percent protein powder by weight. In another example, the nutrients comprise 20 percent hemp-derived protein powder by weight. In some examples, the soil amendment may further include a binding agent. The binding agent may include at least one sugar such as dextrose, fructose, sucrose, and mannitol. In some examples, the binding agent may comprise molasses.

In other examples, the liquid suspension may be lyophilized to obtain a powder that contains the at least one living microorganism. In lyophilized form, the living microorganisms may be dormant, but can be reactivated upon contact with water. A powdered form of the soil amendment may be provided to the soil by applying to the surface of the soil or mixing into the soil. When the soil is watered, the at least one living microorganism may be rehydrated and reactivated. Instead of adding the powder directly to the soil, the powder may be mixed into water and provided to the soil. In another example, a suitable amount of the powder may be compacted to form a pellet, tab, disk or other form. The compacted powder may be applied to the surface of the soil, buried in the soil, or dissolved in the water that the soil is watered with. In yet other examples, the soil amendment may comprise a capsule having a water-soluble shell that encapsulates the at least one microorganism. The water-soluble shell may be biodegradable. The capsule may be applied to the surface of the soil or buried within the soil such that, when the water-soluble shell dissolves, the at least one microorganism is released into the soil. The water-soluble shell may be selected to control the release of the at least one microorganism into the soil In some examples, the water-soluble shell may be selected to degrade such that the at least one living microorganism is released in small amounts over an extended period of time. In other examples, the water-soluble shell may be selected to degrade such that the at least one living microorganism is released quickly over a short period of time.

Once the microorganisms are added to the soil, they may begin to solubilize the insoluble forms of silicon if any are present in the soil. As the silica is solubilized, it may be absorbed by the plant and may provide a number of benefits to the plant. Firstly, the silica may provide pest resistance at the cellular level. Increased silicon content contributes to the development of rigid, silicon-based structures known as phytoliths which may increase the hardness of plant tissues and reduce injuries and damage caused by sap-sucking insects. Secondly, plants with high silicon content may be more tolerant of droughts, freezing, and temperature fluctuations than plants with lower silicon content. This attribute may be particularly useful to Cannabis growers in some climates, including North American climates, where temperatures can drastically fluctuate during harvest, ranging from 30° C. (86° F.) to −5° C. (23° F.) over the course of 24 hours. Thirdly, silica can help to strength cell walls, which leads to greater stalk and stem strength. Wind and rain damage will be minimized thus leading to larger flowers and greater yields. A stronger fibrous main stem translates into the ability to hold more weight or, in the case of Cannabis, bud sites. Fourthly, plants with high silicon content may have improved canopy growth, allows for more space between bud sites and therefore greater light penetration through the canopy reaching the mid-level and low-bud sites. Since light levels improve maturity of buds, plants with greater silicon may have larger flowers and greater crop yields. Furthermore, silica may reduce or prevent transplant shock, a condition that typically arises after a plant is transplanted or sustains damage to its root ball.

The soil amendment may be applied to the soil of any suitable plant. In some examples, the plant is an agricultural crop. In specific examples, the plant is a tomato plant. In other examples, the plant is a Cannabis plant such as an industrial hemp plant or a psychoactive variety of Cannabis.

To increase the quantity of silicon in the soil, the soil amendment may further comprise silica. Adding silica to the soil may provide protect a plant from pests. Soil-borne insects are known to attack plants and the silica may provide a surface layer on the top of the soil which breaks their breeding cycle. Silica's insecticidal properties arises from its absorptive properties; insects have fine waxy membranes that help them to retain water and silica can absorb these membranes, causing the insects to dehydrate and eventually die.

The silica may be crushed or fragmented into small particles to increase the surface area of the particles, thereby increasing the rate at which the silica is solubilized by the at least one living microorganism. The quantity of silica added to the soil may be selected to accommodate the silicon requires of a particular plant. For example, the quantity of silica may be selected to mimic the soil conditions of the ecosystem where a particular plant originates. If the plant is species of Cannabis, the quantity of silica may be selected to replicate the soils of Afghanistan where Cannabis originates. In some examples, the quantity of silica in the soil amendment may be selected such that the soil amendment comprises approximately 5 to 10 percent silica by weight. There are a few different application methods for providing silica to the soil. For example, the silica may be mixed into the soil, the silica may be mixed in water and poured into the soil, or the silica may be applied to the surface of the soil.

In some examples, the soil amendment is a single mixture comprising both the at least one living microorganism and the silica. However, in other examples, the soil amendment comprises two separate mixtures: one comprising the at least one living microorganism and the other comprising the silica.

### Testing the Soil Amendment

The inventors conducted testing to evaluate the effects of the soil amendment on Cannabis indica growth. The inventors grew 25 Cannabis indica plants in a single greenhouse. All 25 plants were propagated from cuttings of a single parent plant.

The inventors divided the 25 plants into a control group comprising 5 plants grown in a standard nutrient soil (Promix®; Mississauga, Canada) and 5 test groups (TG1, TG2, TG3, and TG4), each comprising 5 plants treated with one or a combination of additional treatments as described in Table 1:

TABLE 1
Soil conditions of control group and test groups

| | Base | Silica | Silica | Silica Solubilizing | |
|---|---|---|---|---|---|
| Group | Soil | Dressing | Soil | Microorganisms | Azomite ® |
| Control | Yes | No | No | No | No |
| Group | | | | | |
| TG1 | Yes | Yes | No | No | No |
| TG2 | Yes | Yes | No | Yes | No |
| TG3 | Yes | Yes | Yes | Yes | Yes |
| TG4 | Yes | Yes | Yes | Yes | Yes |

In the chart above, “Base Soil” refers a basic soil used for both the test group and control group. “Fortified Silica Dressing” refers to a layer of silica that has been fractured and wash before being added to the top layer of soil. “Silica Soil” refers to the base soil with a percentage of crushed silica mixed in. Azomite® (Pacific Sprinklers Ltd.; South Slocan, Canada) is an absorptive material that is mixed into the soil to retain nutrients.

“Silica Solubilizing Microorganisms” refers to an aqueous solution comprising:
1) Approximately 1.5 percent by mass of each Pseudomonas putida (Carolina Biological Supply Co.®; Burlington, United States), Pseudomonas montelli (Earth Alive® Clean Technologies; LaSalle, Canada), Bacillus subtilis (Earth Alive® Clean Technologies; LaSalle, Canada), Bacillus amyloliquefaciens (Earth Alive® Clean Technologies; LaSalle, Canada), Glomus intraradices (Earth Alive® Clean Technologies; LaSalle, Canada), Glomus mosseae (Earth Alive® Clean Technologies; LaSalle, Canada), Glomus aggregatum (Earth Alive® Clean Technologies; LaSalle, Canada), Glomus etunicatum (Earth Alive® Clean Technologies; LaSalle, Canada), Glomus clarum (Earth Alive® Clean Technologies; LaSalle, Canada), Glomus deserticola (Earth Alive® Clean Technologies; LaSalle, Canada), Gigaspora margarita (Earth Alive® Clean Technologies; LaSalle, Canada), Paraglomus brasilianum (Earth Alive® Clean Technologies; LaSalle, Canada), Glomus monosporum (Earth Alive® Clean Technologies; LaSalle, Canada), Rhizopogon villosullus (Earth Alive® Clean Technologies; LaSalle, Canada), Rhizopogon luteolus (Earth Alive® Clean Technologies; LaSalle, Canada), Rhizopogon amylopogon (Earth Alive® Clean Technologies; LaSalle, Canada), Rhizopogon fulvigleba (Earth Alive® Clean Technologies; LaSalle, Canada), Pisolithus tinctorius (Earth Alive® Clean Technologies; LaSalle, Canada), Laccaria bicolor (Earth Alive® Clean Technologies; LaSalle, Canada), Laccaria laccata (Earth Alive® Clean Technologies; LaSalle, Canada), Suillus granulates (Earth Alive® Clean Technologies; LaSalle, Canada), Suillus punctatpies (Earth Alive® Clean Technologies; LaSalle, Canada), Bacillus pumilus (Earth Alive® Clean Technologies; LaSalle, Canada), Paenibacillus macerans (Earth Alive® Clean Technologies; LaSalle, Canada), Bacillus firmus (Earth Alive® Clean Technologies; LaSalle, Canada), and Paenibacillus macqariensis (Earth Alive® Clean Technologies; LaSalle, Canada);
2) 20 percent by mass hemp-derived protein powder (Karma Co-Operative; Toronto, Canada);
3) 20 percent by mass activated yeast (Karma Co-op; Toronto, Canada); and
4) 2 percent by mass dextrose (Karma Co-op; Toronto, Canada).

After 6 months, the inventors evaluated the plants by measuring plant height, canopy height, mature canopy height, canopy circumference, number of apex buds, number of mid buds, number of low buds, total number of buds, main stem circumference, main feeder branches off primary, secondary flower branches, and number of larf buds. The results are recorded in Table 2 below:

TABLE 2
Measurements of plants after 6 months

| | Control | | | | | Test |
|---|---|---|---|---|---|---|
| | Group | TG1 | TG2 | TG3 | TG4 | Group |
| | Aver- | Aver- | Aver- | Aver- | Aver- | Aver- |
| | age | age | age | age | age | age |
| Plant | 43.6 | 49.6 | 43.6 | 47.4 | 46.9 | 46.9 |
| Height | | | | | | |
| (inches) | | | | | | |
| Canopy | 23.6 | 25.2 | 20.8 | 25 | 24.4 | 23.9 |
| Height | | | | | | |
| (inches) | | | | | | |
| Mature | 17.6 | 24.8 | 16.4 | 19.6 | 19.8 | 20.2 |
| Canopy | | | | | | |
| Height | | | | | | |
| (inches) | | | | | | |
| Canopy | 34 | 27.4 | 34.8 | 35 | 29.4 | 31.7 |
| Circumference | | | | | | |
| (inches) | | | | | | |
| No. of | 13.2 | 13.8 | 15.2 | 22.6 | 22.6 | 18.6 |
| Apex buds | | | | | | |
| No. of | 44.4 | 130.6 | 98.6 | 105.6 | 97.2 | 108.0 |
| Mid Buds | | | | | | |
| No. of | 17.8 | 34 | 20.6 | 29 | 23.2 | 26.7 |
| Low Buds | | | | | | |
| Total Buds | 75.4 | 178.4 | 134.4 | 157.2 | 143 | 153.3 |
| Main Stem | 4.8 | 4.8 | 4.8 | 5.25 | 6.4 | 5.3 |
| Circumference | | | | | | |
| (inches) | | | | | | |
| No. of | 8.6 | 12.2 | 11 | 12.75 | 13.8 | 12.4 |
| Secondary | | | | | | |
| Flower | | | | | | |
| Branches | | | | | | |
| No. of | 59.2 | 26 | 1 | 20.4 | 11.6 | 17.3 |
| Larf Buds | | | | | | |

Table 3 shows a comparison of the average values for the control group to the average values for each of the test groups.

TABLE 3
Comparisons of average values for control
group to average values of test groups

| | Control | | | | | Test |
|---|---|---|---|---|---|---|
| | Group | TG1 | TG2 | TG3 | TG4 | Group |
| | Aver- | Aver- | Aver- | Aver- | Aver- | Aver- |
| | age | age | age | age | age | age |
| Plant | 100% | 113.8% | 100% | 108.7% | 107.6% | 107.5% |
| Height | | | | | | |
| (inches) | | | | | | |
| Canopy | 100% | 106.8% | 88.1% | 105.9% | 103.4% | 101.1% |
| Height | | | | | | |
| (inches) | | | | | | |
| Mature | 100% | 140.9% | 93.2% | 111.4% | 112.5% | 114.5% |
| Canopy | | | | | | |
| Height | | | | | | |
| (inches) | | | | | | |
| Canopy | 100% | 80.6% | 120.4% | 102.9% | 86.5% | 93.1% |
| Circumference | | | | | | |
| (inches) | | | | | | |
| No. of | 100% | 294.1% | 222.1% | 237.8% | 218.9% | 243.2% |
| Apex buds | | | | | | |
| No. of | 100% | 191% | 115.7% | 162.9% | 130.3% | 150% |
| Mid Buds | | | | | | |
| No. of | 100% | 236.6% | 178.2% | 208.5% | 189.7% | 203.2% |
| Low Buds | | | | | | |
| Total Buds | 100% | 105.1% | 102.9% | 110.9% | 110.5% | 107.3% |
| Main Stem | 100% | 100% | 100% | 109.4% | 133.3% | 110.7% |
| Circumference | | | | | | |
| (inches) | | | | | | |
| No. of | 100% | 141.9% | 127.9% | 148.3% | 160.5% | 144.6% |
| Secondary | | | | | | |
| Flower | | | | | | |
| Branches | | | | | | |
| No. of | 100% | 43.9% | 18.6% | 34.5% | 19.6% | 29.1% |
| Larf Buds | | | | | | |

As described in the tables above, the inventors found significant differences between each test group and the control group. The test groups outperformed the control group in nearly every characteristic measured. On average, the test group plants were taller with taller canopy heights, although the canopy circumference for some of the test groups were narrower than the control group average. The stem circumference was also larger for the test groups on average than the control group. Furthermore, the test groups had a higher number of apex buds, mid buds and low buds on average as compared to the control group, while having a smaller number of larf buds.

After 6 months, the inventors further analyzed the plants to measure the chemical content of the buds. Specifically, the following compounds were measured: tetrahydrocannabinol (THC), Cannabidiol (CBD), Cannabigerol (CBG), Cannabidivarin (CBDV), Tetrahydrocannabivarin (THCV), Cannabichromevarin (CBCV), Cannabinol (CBN), Cannabichromene (CBC), Cannabicyclol (CBL), and cannabitriol (CBT). The weight concentrations (% w/w) of each compound, as measured in the plants, is described below in Table 4:

TABLE 4
Weight concentrations (% w/w) of
relevant compounds in plant buds

| | Control | | | | | Test |
|---|---|---|---|---|---|---|
| | Group | TG1 | TG2 | TG3 | TG4 | Group |
| | Aver- | Aver- | Aver- | Aver- | Aver- | Aver- |
| (% w/w) | age | age | age | age | age | age |
| THC | 7.991 | 10.789 | 8.890 | 7.794 | 8.028 | 8.875 |
| CBD | 0.017 | 0.026 | 0.0022 | 0.021 | 0.020 | 0.022 |
| CBG | 0.365 | 0.801 | 0.571 | 0.408 | 0.398 | 0.545 |
| CBDV | 0.009 | 0.006 | 0.014 | 0.013 | 0.010 | 0.011 |
| THCV | 0.123 | 0.198 | 0.148 | 0.135 | 0.133 | 0.154 |
| CBCV | 0.008 | 0.011 | 0.009 | 0.008 | 0.008 | 0.009 |
| CBN | 0.011 | 0.019 | 0.015 | 0.014 | 0.014 | 0.015 |
| CBC | 0.106 | 0.190 | 0.134 | 0.122 | 0.128 | 0.144 |
| CBL | 0.298 | 0.509 | 0.376 | 0.292 | 0.314 | 0.373 |
| CBT | 0.184 | 0.224 | 0.159 | 0.117 | 0.141 | 0.161 |

Table 5 shows a comparison of the average values for the control group to the average values for each of the test groups:

TABLE 5
Comparison of average values for control
group to average values for test groups

| | Control | | | | | Test |
|---|---|---|---|---|---|---|
| | Group | TG1 | TG2 | TG3 | TG4 | Group |
| | Aver- | Aver- | Aver- | Aver- | Aver- | Aver- |
| (% w/w) | age | age | age | age | age | age |
| THC | 100% | 135% | 111% | 98% | 100% | 111% |
| CBD | 100% | 152% | 127% | 121% | 119% | 129% |
| CBG | 100% | 219% | 156% | 112% | 109% | 149% |
| CBDV | 100% | 62% | 164% | 145% | 108% | 120% |
| THCV | 100% | 161% | 120% | 110% | 108% | 125% |
| CBCV | 100% | 143% | 119% | 105% | 104% | 118% |
| CBN | 100% | 167% | 129% | 122% | 118% | 134% |
| CBC | 100% | 180% | 127% | 115% | 121% | 136% |
| CBL | 100% | 171% | 126% | 98% | 105% | 125% |
| CBT | 100% | 122% | 87% | 64% | 77% | 87% |

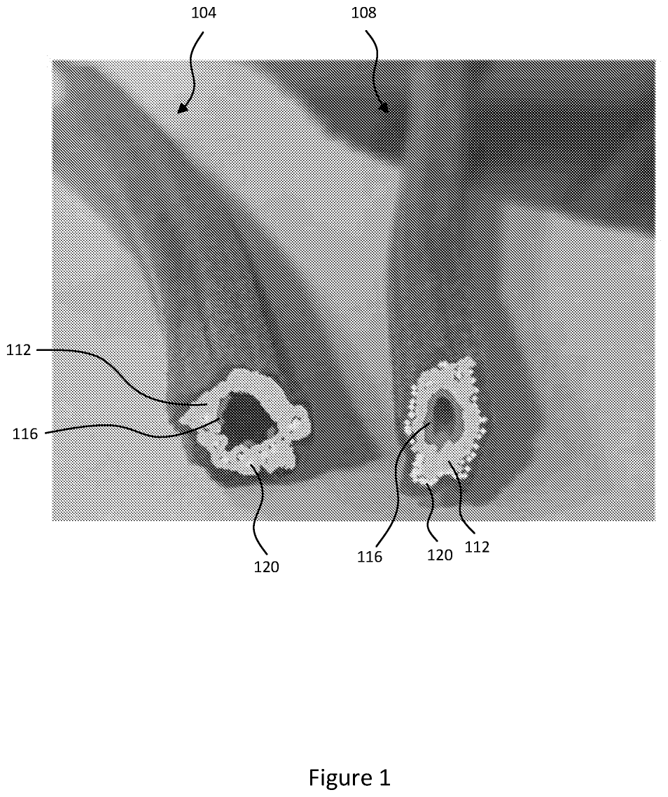

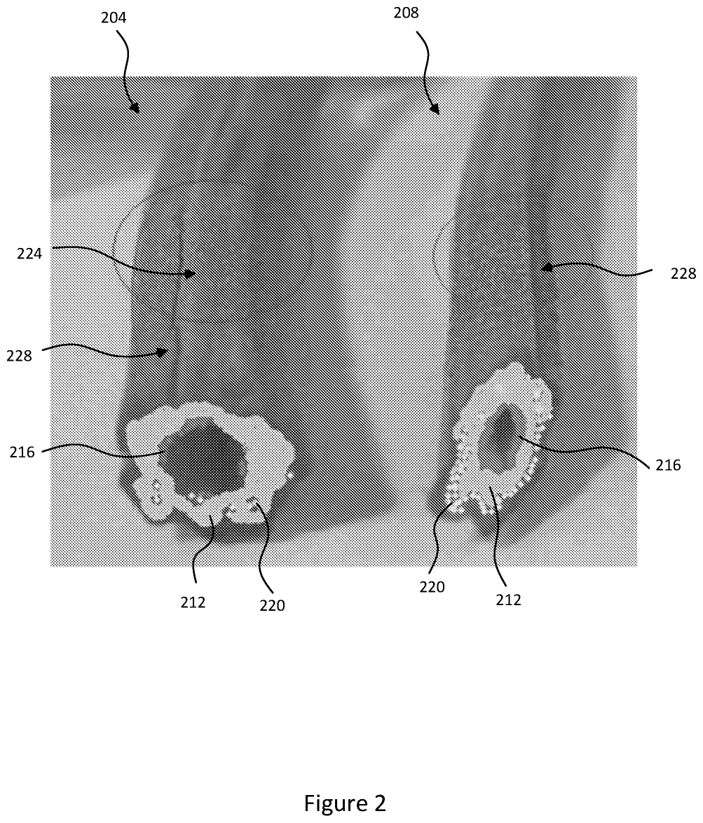

The stalks of the plants were further examined for vascular structures. FIG. 1 shows a photograph of cross-section of a stalk of one plant from the test group 104 and one plant from the control group 108. In the image, phloem is indicated at 112, xylem is indicated at 116, and xylem bundles are indicated at 120. The stem from the test group 104 is not only wider, it also has a greater ratio of phloem to xylem as compared to the control group 108. Phloem is more fibrous than xylem and therefore indicates a stronger stem capable of supporting more weight and resisting the elements. FIG. 2 shows a photograph of a cross-section of a stalk from another plant of the test group 204 and another plant from the control group 208. In the image, phloem is indicated at 212, xylem is indicated at 216, xylem bundles are indicated at 220, cystoliths are indicated generally at 224, epidermal fissures are indicated at 228. Similar to FIG. 1, the stem from the test group 204 is wider and has a greater ratio of phloem to xylem than the control group 208. Furthermore, the stem from the test group 204 has cystoliths, which are known to deter pests. Both the test group 204 and the control group 208 have epidermal fissures.

The plants were observed for algal growth and mold after 3 months and again after 6 months. All 5 plants in the control group were observed to have algal growth and none of the plants in the test groups were observed to have algal growth.

The many features and advantages of the present disclosure will now be apparent from the detailed specification. For example, the presently disclosed soil amendment may improve pest resistance by providing a physical barrier in the soil and encouraging the growth of plant structures that deter feeding by insects. Furthermore, the soil amendment may encourage nutrient absorption and vascular development in plants which enables the plants to better withstand drought, frost, winds, temperature fluctuations, and other environmental conditions. Overall, these benefits contribute to increased plant growth and crop yields, particularly in Cannabis plants. Since the microorganisms included in the soil amendment are able to multiply in the soil, the soil amendment need only be applied once or infrequently in order to attain the desired benefits.

Since numerous modifications and changes will readily occur to those skilled in the art, it is not desired to limit the invention to the exact construction and operation illustrated and described, and accordingly all suitable modifications and equivalents may be resorted to, falling within the scope of the invention.

1. A soil amendment comprising at least one living microorganism, wherein said at least one microorganism is capable of solubilizing silicate.

2. The soil amendment of claim 1, wherein the at least one living microorganism is selected from a group consisting of Pseudomonas putida, Pseudomonas montelli, Bacillus subtilis, Bacillus amyloliquefaciens, Glomus intraradices, Glomus mosseae, Glomus aggregatum, Glomus etunicatum, Glomus clarum, Glomus deserticola, Gigaspora margarita, Paraglomus brasilianum, Glomus monosporum, Rhizopogon villosullus, Rhizopogon luteolus, Rhizopogon amylopogon, Rhizopogon fulvigleba, Pisolithus tinctorius, Laccaria bicolor, Laccaria laccata, Suillus granulates, Suillus punctatpies, Bacillus pumilus, Paenibacillus macerans, Bacillus firmus, and Paenibacillus macqariensis, and combinations thereof.

3. The soil amendment of claim 1, wherein the at least one living microorganism comprises Pseudomonas putida, Pseudomonas montelli, Bacillus subtilis, Bacillus amyloliquefaciens, Glomus intraradices, Glomus mosseae, Glomus aggregatum, Glomus etunicatum, Glomus clarum, Glomus deserticola, Gigaspora margarita, Paraglomus brasilianum, Glomus monosporum, Rhizopogon villosullus, Rhizopogon luteolus, Rhizopogon amylopogon, Rhizopogon fulvigleba, Pisolithus tinctorius, Laccaria bicolor, Laccaria laccata, Suillus granulates, Suillus punctatpies, Bacillus pumilus, Paenibacillus macerans, Bacillus firmus, and Paenibacillus macqariensis.

4. The soil amendment of claim 1, further comprising an aqueous solution, wherein the at least one living microorganism is suspended in the aqueous solution.

5. The soil amendment of claim 1, further comprising at least one nutrient.

6. The soil amendment of claim 1, further comprising a binding agent.

7. The soil amendment of claim 4, wherein the aqueous solution is lyophilized to obtain a powder.

8. The soil amendment of claim 7, wherein the powder is compounded into a pellet.

9. The soil amendment of claim 7, wherein the powder is encased in a water-soluble capsule.

10. The soil amendment of claim 9, wherein the water-soluble capsule is selected to control the release of the at least one microorganism.

11. The soil amendment of any ono of claims 1 to 4 claim 1, further comprising silica.

12. The soil amendment of claim 11, wherein the silica comprises insoluble silicate.

13. The soil amendment of claim 11, wherein the at least one microorganism and the silica increase the potency of any added nutrients.

14. The use of the soil amendment of claim 1 to improve growth of plants.

15. The use of claim 14, wherein the use is further to improve vascular development in plants.

16. The use of claim 14, wherein the use is further to increase nutrient absorption by plants.

17. The use of claim 14, wherein the use is further to improve frost resistance in plants.

18. The use of claim 14, wherein the use is further to improve drought resistance in plants.

19. The use of claim 14, wherein the use is further to improve pest resistance in plants.

20. The use of claim 14, wherein the use is further to reduce transplant shock in plants.

21. The use of claim 14, wherein the use is further to improve growth of tomato plants.

22. The use of claim 14, wherein the use is further to improve growth of Cannabis plants.

23. The use of claim 14, wherein the use is further to improve vascular development in Cannabis plants.

24. The use of claim 14, wherein the use is further to increase nutrient absorption by Cannabis plants.

25. The use of claim 14, wherein the use is further to improve frost resistance in Cannabis plants.

26. The use of claim 14, wherein the use is further to improve drought resistance in Cannabis plants.

27. The use of claim 14, wherein the use is further to improve pest resistance in Cannabis plants.

28. The use of claim 14, wherein the use is further to increase the number of bud sites in Cannabis plants.

29. The use of claim 14, wherein the use is further to reduce the number of larf buds in Cannabis plants.

30. The use of claim 14, wherein the use is further to increase crop yield in Cannabis plants.

31. The use of claim 14, wherein the use is further to reduce transplant shock in Cannabis plants.

32. The use of claim 21, wherein the use is further to improve growth of hemp plants.
